(19) United States
(12) Patent Application Publication
(10) Pub. No.: US 2022/0104715 A1
(43) Pub. Date: Apr. 7, 2022

(54) BLOOD-PRESSURE MEASUREMENT DEVICE, MODEL SETTING DEVICE, AND BLOOD-PRESSURE MEASUREMENT METHOD

(71) Applicant: SHARP KABUSHIKI KAISHA, Sakai City, Osaka (JP)

(72) Inventors: RIEKO OGAWA, Sakai City, Osaka (JP); YOSHIHISA ADACHI, Sakai City, Osaka (JP); YOSHIFUMI IWAI, Sakai City, Osaka (JP); YUKI EDO, Sakai City, Osaka (JP); RYOTA TOMIZAWA, Sakai City, Osaka (JP)

(21) Appl. No.: 17/427,044

(22) PCT Filed: Jan. 29, 2020
(86) PCT No.: PCT/JP2020/003185

§ 371 Date: Jul. 29, 2021

(30) Foreign Application Priority Data
Feb. 1, 2019 (JP) 2019-017189

(51) Int. Cl. A61B 5/021; A61B 5/024; A61B 5/00; G16H 40/63; G16H 30/20

(52) U.S. Cl. CPC A61B 5/02108; A61B 5/02416; A61B 5/7278; A61B 5/0022; A61B 5/02125; G16H 40/63; G16H 30/20; A61B 5/02116; A61B 5/7267

(57) ABSTRACT

A blood-pressure measuring device that measures a first blood pressure of a living body on the basis of a pulse wave of the living body, the blood-pressure measuring device includes: a pulse-wave acquiring unit configured to acquire at least one pulse wave; a pulse-wave-parameter calculator configured to calculate at least one pulse wave parameter based on the at least one pulse wave; an attribute-information acquiring unit configured to acquire attribute information that relates to a vascular condition of the living body; and an attribute classifier configured to classify an attribute of the living body, wherein the blood-pressure measuring device is communicably connected to a model storage storing in advance one or more blood-pressure-estimation models, and the blood-pressure measuring device further comprises a first-blood-pressure measuring unit configured to calculate the first blood pressure based on the at least one pulse wave parameter by using the one or more blood-pressure-estimation models.

### TECHNICAL FIELD

An aspect of the present disclosure relates to a blood-pressure measuring device that measures the blood pressure of a living body on the basis of the pulse wave of the living body. The present application claims priority from Japanese Patent Application No. 2019-17189, filed on Feb. 1, 2019, the content of which is hereby incorporated by reference into this application.

### BACKGROUND ART

Various techniques have been recently proposed for measuring the blood pressure of a living body (e.g., a subject). By way of example, Patent Literature 1 discloses a technique for measuring (more strictly, estimating) the blood pressure of a living body easily. To be specific, the technique in Patent Literature 1 uses a model (computational expression) that is preformulated in accordance with the age (actual age) and sex of a living body, to estimate the blood pressure of the living body.

### CITATION LIST

### Patent Literatures

Patent Literature 1: Japanese Patent Application Laid-Open No. 2010-220690

Patent Literature 2: Japanese Patent Application Laid-Open No. 2002-238867

Patent Literature 3: Japanese Patent Application Laid-Open No. 2015-40839

Patent Literature 4: Japanese Patent Application Laid-Open No. 2009-086901

### SUMMARY OF INVENTION

### Technical Problem

Unfortunately, how to specifically measure the blood pressure of a living body with high accuracy needs to be improved, as described below. It is an object of one aspect of the present disclosure to measure the blood pressure of a living body more accurately than before.

### Solution to Problem

To solve the above problem, one aspect of the present disclosure provides a blood-pressure measuring device that measures a first blood pressure of a living body on the basis of the pulse wave of the living body. The blood-pressure measuring device includes the following: a pulse-wave acquiring unit that acquires at least one pulse wave in a predetermined region on the body surface of the living body; a pulse-wave-parameter calculator that calculates at least one pulse wave parameter based on the at least one pulse wave; an attribute-information acquiring unit that acquires attribute information that relates to the vascular condition of the living body; and an attribute classifier that classifies the attribute of the living body in accordance with the attribute information. The blood-pressure measuring device is communicably connected to a model storage storing in advance one or more blood-pressure-estimation models that are used for estimating the first blood pressure based on the result of classification of the attribute. The blood-pressure measuring device further includes a first-blood-pressure measuring unit that calculates the first blood pressure based on the at least one pulse wave parameter by using the one or more blood-pressure-estimation models based on the result of classification of the attribute.

To solve the above problem, one aspect of the present disclosure provides a model setting device communicably connected to a blood-pressure measuring device that measures a first blood pressure of the living body on the basis of the pulse wave of the living body. The model setting device includes the following: a second-blood-pressure measuring unit that measures a second blood pressure of the living body; a pulse-wave acquiring unit that acquires at least one pulse wave in a predetermined region on the body surface of the living body; a pulse-wave-parameter calculator that calculates at least one pulse wave parameter based on the at least one pulse wave; an attribute-information acquiring unit that acquires attribute information that relates to the vascular condition of the living body; and an attribute classifier that classifies the attribute of the living body in accordance with the attribute information. The model setting device is communicably connected to a model storage capable of storing one or more blood-pressure-estimation models that are used for estimating the first blood pressure based on the result of classification of the attribute. The model setting device further includes a model creating unit that creates the one or more blood-pressure-estimation models based on the at least one pulse wave parameter and the second blood pressure, and stores the one or more blood-pressure-estimation models in the model storage.

To solve the above problem, one aspect of the present disclosure provides a blood-pressure measurement method using a blood-pressure measuring device that measures a first blood pressure of a living body on the basis of the pulse wave of the living body. The method includes the following steps: acquiring at least one pulse wave in a predetermined region on the body surface of the living body; calculating at least one pulse wave parameter based on the at least one pulse wave; acquiring attribute information that relates to the vascular condition of the living body; and classifying the attribute of the living body in accordance with the attribute information. The blood-pressure measuring device is communicably connected to a model storage storing in advance one or more blood-pressure-estimation models that are used for estimating the first blood pressure based on the result of classification of the attribute. The method further includes a step of calculating the first blood pressure based on the at least one pulse wave parameter by using the one or more blood-pressure-estimation models based on the result of classification of the attribute.

### Advantageous Effect of Invention

The blood-pressure measuring device according to the aspect of the present disclosure can measure the blood pressure of a living body more accurately than before. The blood-pressure measurement method according to the aspect of the present disclosure brings a similar effect. The model setting device according to the aspect of the present disclosure brings a similar effect.

### BRIEF DESCRIPTION OF DRAWINGS

FIG. 1 is a functional block diagram illustrating the configuration of main components of a blood-pressure measuring device according to a first embodiment.

FIG. 2 illustrates an example process step performed by a facial-image divider.

FIG. 3(a) illustrates an example acceleration pulse waveform obtained from a subject of low vascular age. FIG. 3(b) illustrates an example acceleration pulse waveform obtained from a subject of high vascular age.

FIG. 4 is a table illustrating an example result obtained by classifying a plurality of subjects on the basis on the amount of a waveform characteristic.

FIG. 5(a) is a graph illustrating an example relationship between exact blood pressure and predicted blood pressure in a common model. FIG. 5(b) is a graph illustrating an example relationship between exact blood pressure and predicted blood pressure in an attribute-specific model.

FIG. 6 illustrates an example process for a model evaluating unit to set a measurement model.

FIG. 7 is a flowchart illustrating how to set a measurement model.

FIG. 8 is a graph illustrating an example power spectrum of a pulse wave signal.

FIG. 9 is a graph illustrating an example relationship between blood pressure and PWV for each of subjects having different attributes.

FIG. 10 is a functional block diagram illustrating the configuration of main components of a blood-pressure measuring device according to a second embodiment.

FIG. 11 is a table illustrating average blood pressures and attributes by way of example.

FIG. 12 illustrates example manual input of a blood pressure.

FIG. 13(a) is a table illustrating example information containing the subject's name and attribute associated with each other. FIG. 13(b) is a table illustrating example information containing the subject's ID and attribute associated with each other.

FIG. 14 is a flowchart illustrating an example process performed by the blood-pressure measuring device.

FIG. 15 is a scatter diagram of average blood pressure and pulse pressure. FIG. 15 is also a table illustrating example attributes corresponding to the average blood pressure and pulse pressure.

FIG. 16 illustrates a modification using the cloud.

### DESCRIPTION OF EMBODIMENTS

### First Embodiment

The following describes a blood-pressure measuring device 1 according to a first embodiment. For convenience, components of the same functions as those described in the first embodiment will be denoted by the same sings and will not be elaborated upon in the subsequent embodiments. Descriptions similar to publicly known techniques will be omitted as necessary. Configurations in the individual drawings are illustrative for convenience in description; so are numeric values in the Description.

Outline of Blood-Pressure Measuring Apparatus 1

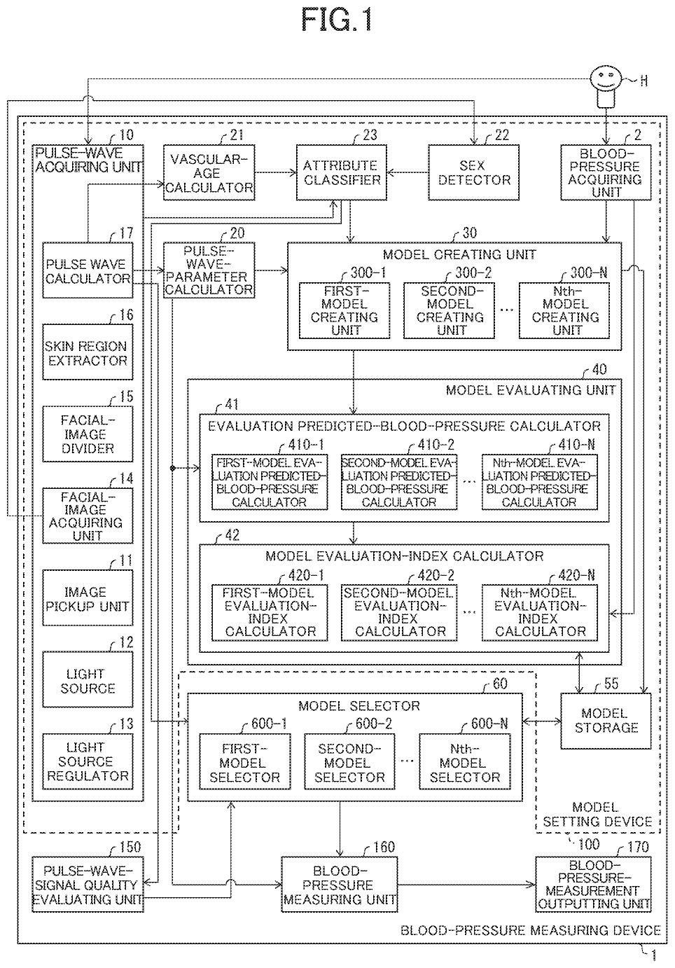

FIG. 1 is a functional block diagram illustrating the configuration of main components of the blood-pressure measuring device 1 according to the first embodiment. The blood-pressure measuring device 1 measures the blood pressure of a subject H or living body (hereinafter merely referred to as blood pressure) on the basis of the pulse wave of the subject H. To be specific, the blood-pressure measuring device 1 measures the blood pressure by using a blood-pressure-measurement model (hereinafter also merely referred to as a measurement model) that is set by a model setting device 100, which will be described below. It is noted that in the Description, a blood-pressure-estimation model, described later on, is also merely referred to as an estimation model. It is also noted that a measurement model and an estimation model are also merely referred to as a model generically.

The following describes the blood-pressure measuring device 1, which is a contactless blood-pressure measuring device (a blood-pressure measuring device capable of measuring the blood pressure without contacting the subject H). The first embodiment describes an instance where the subject H is a human. The blood-pressure measuring device 1 measures the blood pressure by using a predetermined region on the body surface of the subject H as a region of interest (ROI). The following describes an instance where the ROI is the face. In the Description, the face of the subject H is also merely referred to as the face. This holds true for the other indications.

The blood-pressure measuring device 1 includes the model setting device 100, a model selector 60, a pulse-wave-signal quality evaluating unit 150, a blood-pressure measuring unit 160 (first-blood-pressure measuring unit), and a blood-pressure-measurement outputting unit 170. The model setting device 100 includes a blood-pressure acquiring unit 2 (second-blood-pressure measuring unit), a pulse-wave acquiring unit 10, a pulse-wave-parameter calculator 20, a vascular-age calculator 21, a sex detector 22, an attribute classifier 23, a model creating unit 30, a model evaluating unit 40, and a model storage 55.

FIG. 1 illustrates an instance where the model setting device 100 is disposed inside the blood-pressure measuring device 1. The model setting device 100 can be disposed outside the blood-pressure measuring device 1 (see a second embodiment, which will be described later on).

The blood-pressure acquiring unit 2 measures the blood pressure of the subject H. The blood-pressure acquiring unit 2 is a contact blood-pressure gauge (e.g., a cuff blood-pressure gauge). The blood pressure (hereinafter, a BPm) measured by the blood-pressure acquiring unit 2 is used as test data (or training data) in the model setting device 100. That is, the BPm is used for the model setting device 100 (more specifically, the model evaluating unit 40) to set a measurement model. The BPm is also used for the model creating unit 30 to create one or more estimation models.

The blood-pressure acquiring unit 2 outputs the BPm to the model creating unit 30 and model evaluating unit 40 (more specifically, to a model evaluation-index calculator 42, which will be described below). Here, a final blood pressure measurement (P, which will be described later on) measured by the blood-pressure measuring device 1 is also referred to as a first blood pressure. In addition, the BPm is also referred to as a second blood pressure in order to distinguish the BPm from the first blood pressure. As described later on, the P is measured (calculated) by the blood-pressure measuring unit 160.

Pulse-Wave Acquiring Unit 10

The pulse-wave acquiring unit 10 acquires the pulse wave in a ROI (e.g., the face). The pulse-wave acquiring unit 10 includes an image pickup unit 11, a light source 12, a light source regulator 13, a facial-image acquiring unit 14, a facial-image divider 15, a skin region extractor 16, and a pulse wave calculator 17.

The image pickup unit 11 is a camera including an image sensor and a lens. The image sensor may be a complementary metal-oxide semiconductor (CMOS) image sensor or a charge-coupled device (CCD) image sensor, for instance. The image pickup unit 11 captures an image of the subject H multiple times and outputs the captured image of the subject H (hereinafter, referred to as a subject image) to the facial-image acquiring unit 14 at a predetermined frame rate (i.e., at predetermined time intervals). The frame rate is 300 fps (frames per second) by way of example.

The image pickup unit 11 may include a publicly known color filter. This color filter preferably has optical properties suitable for observing fluctuations in blood volume. Possible suitable examples of the color filter include (i) a red-blue-green-cyan (RGBCy) color filter and (ii) a red-blue-green-infrared (RGBIR) color filter. Alternatively, the color filter may be a Bayer-arrangement color filter. As such, the image pickup unit 11 may be an RGB camera or an IR camera.

The light source 12 emits light to the subject H when the image pickup unit 11 captures an image of the subject H. The light source regulator 13 regulates the light source 12. By way of example, the light source regulator 13 preferably regulates the light source in such a manner that a pulse transit time (an example pulse wave parameter) between regions used in a measurement model established by the model setting device 100 can be calculated accurately.

To be specific, the light source regulator 13 regulates the light source 12 in such a manner that a pulse wave of predetermined signal quality in a pertinent region can be detected. Herein, a pulse wave of predetermined signal quality refers to, for instance, a pulse wave having a high single-to-noise ratio (SNR). To be more specific, the light source regulator 13 regulates at least one of (i) the amount of light from the light source 12, (ii) the light spectrum of the light source 12, and (iii) the angle of irradiation to the skin of the subject H.

The pulse-wave acquiring unit 10 does not necessarily have to include the light source 12 and light source regulator 13. When the light source 12 and the light source regulator 13 are not provided, the image pickup unit 11 may use only ambient light to capture an image of the subject H.

The facial-image acquiring unit 14 extracts a facial region of the subject H from the subject image captured by the image pickup unit 11. The facial-image acquiring unit 14 acquires an image that has undergone facial-region extraction, as a facial image (an image on which the face of the subject H is appearing). The facial image is an example image including an image of the ROI. By way of example, the facial-image acquiring unit 14 may perform face tracking on a moving image (a moving image consisting of a plurality of subject images) on which the subject is appearing, to extract a facial region for each predetermined frame of the moving image.

Nevertheless, the facial-image acquiring unit 14 can extract the facial region without necessarily performing face tracking. For instance, the image pickup unit 11 may capture a subject image, (i) with the face of the subject H falling within a predetermined frame, and (ii) with the face and image pickup unit 11 fixed. This configuration, which can prevent facial blurring in the subject image, requires no face tracking.

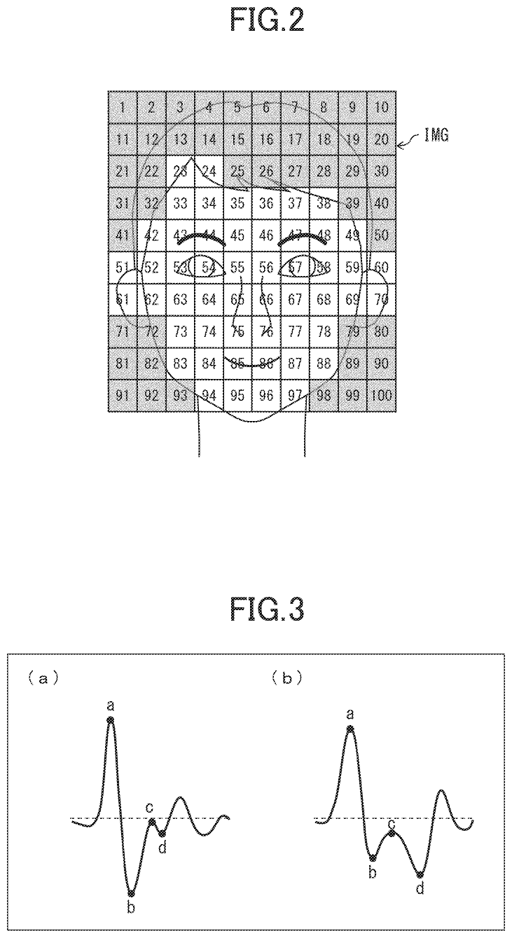

The facial-image divider 15 divides the facial image extracted by the facial-image acquiring unit 14 into a plurality of regions (partial regions). For convenience in description, the facial image is hereinafter referred to as an IMG. FIG. 2 illustrates an example process step performed by the facial-image divider 15. FIG. 2 illustrates an example IMG that has been divided by the facial-image divider 15. The IMG in FIG. 2 is an example facial image on which the face looking straight ahead is appearing.

In the example of FIG. 2, the facial-image divider 15 divides the IMG into tenth vertically and horizontally (e.g., into tenth equally in both directions). That is, the facial-image divider 15 divides the IMG into 100 partial regions (partial regions 1 to 100). Herein, how the facial-image divider 15 divides the IMG is not limited to the example in FIG. 2. For instance, the size of each partial region does not necessarily have to be the same.

The skin region extractor 16 extracts a skin region (a region on which at least part of the skin is appearing) from each partial region. The skin region refers to a region in which the skin is not completely covered with an object (e.g., hair or sunglasses). The skin region can be expressed as a region in which a pulse wave can be detected (calculated). In the example of FIG. 2, each skin region is a region not shaded among the partial regions. In the example of FIG. 2, the skin region extractor 16 extracts 52 skin regions (52 locations) from among the 100 partial regions.

The pulse wave calculator 17 calculates a pulse wave (more strictly, a pulse wave signal) for each of the skin regions extracted by the skin region extractor 16. The pulse wave calculator 17 may calculate the pulse waves through a publicly known method (e.g., a method using independent component analysis). By way of example, the pulse wave calculator 17 may calculate a single pulse wave when the facial-image divider 15 is not provided. The pulse wave calculator 17 needs to calculate at least one pulse wave.

The pulse-wave-parameter calculator 20 calculates a pulse wave parameter on the basis of the pulse wave in each skin region calculated by the pulse wave calculator 17. A pulse wave parameter in the Description generically refers to an explanatory variable (also called an independent variable) that is used in blood pressure measurement (calculation) based on a measurement model. The pulse-wave-parameter calculator 20 needs to calculate at least one pulse wave parameter on the basis of the at least one pulse wave.

An example of the pulse wave parameter usable is a pulse transit time (PTT) between the skin regions. Accordingly, the pulse-wave-parameter calculator 20 in the first embodiment uses a publicly known method to calculate a PTT on the basis of the pulse wave in each skin region. It is noted that the PTT between Region A (any one skin region) and Region B (another skin region different from Region B) is also expressed as PTT (A-B). For instance, the PTT between the regions 23 and 24 in FIG. 2 is expressed as PTT (23-24).

In the example of FIG. 2, the pulse-wave-parameter calculator 20 selects combinations of any two skin regions from among the 52 skin regions. That is, the pulse-wave-parameter calculator 20 selects ₅₂C₂=1326 combinations in total. The pulse-wave-parameter calculator 20 then calculates the PTT for each combination. The pulse-wave-parameter calculator 20 thus calculates 1326 different PTTs, that is, PTT (23-24) to PTT (96-97).

Another example of the pulse wave parameter usable is the amount of a waveform characteristic of the pulse wave in each skin region. Accordingly, the pulse-wave-parameter calculator 20 may calculate the amount of a waveform characteristic through a publicly known method. By way of example, the amount of a waveform characteristic may be calculated based on (i) a pulse waveform, (ii) a velocity pulse waveform (a waveform obtained by performing differentiation on a pulse wave signal one time), or (iii) an acceleration plethysmographic waveform (a waveform obtained by performing differentiation on a pulse wave signal two times).

By way of example, the pulse-wave-parameter calculator 20 may derive an acceleration plethysmographic waveform from a pulse wave signal, followed by analyzing the acceleration plethysmographic waveform to calculate the amount of a waveform characteristic. FIG. 3, described later on, illustrates example acceleration plethysmographic waveforms. To be specific, FIG. 3 shows Points a to d as below, each of which indicates a characteristic point of each acceleration plethysmographic waveform.
Characteristic Point a is a first maximum point of each acceleration plethysmographic waveform.
Characteristic Point b is a first minimum point of each acceleration plethysmographic waveform.
Characteristic Point c is a second maximum point of each acceleration plethysmographic waveform.
Characteristic Point d is a second minimum point of each acceleration plethysmographic waveform.

For easy description, the amplitude at Characteristic Point a of each acceleration plethysmographic waveform is hereinafter simply referred to as Amplitude a (or merely referred to as “a”). This holds true for Characteristic Points b to d.

By way of example, the pulse-wave-parameter calculator 20 may calculate (i) each amplitude (a to d) as the amount of a waveform characteristic. Alternatively, the pulse-wave-parameter calculator 20 may calculate the ratio of each amplitude (e.g., b/a) as the amount of a waveform characteristic. Alternatively, the pulse-wave-parameter calculator 20 may calculate the time difference between the characteristic points (e.g., the time difference between Characteristic Points a and b) as the amount of a waveform characteristic.

Vascular-Age Calculator 21

The vascular-age calculator 21 calculates the vascular age of the subject H on the basis of a pulse wave calculated by the pulse wave calculator 17. For instance, the vascular-age calculator 21 calculates the vascular age by analyzing the pulse wave. This vascular-age calculation may use a publicly known method (see Patent Literature 2 for instance).

Information indicating the vascular age (vascular-age information) is example information about the attribute of the subject H (hereinafter, merely referred to as attribute information). In the Description, attribute information generically refers to information relating to the vascular condition of the subject H (vascular-condition-related information). This vascular-condition-related information is example information relating to a unique nature of the subject H (more specifically, the constitution of the subject H). In the Description, a functional unit that acquires the attribute information is generically referred to as an attribute-information acquiring unit. The vascular-age calculator 21 is thus an example of the attribute-information acquiring unit.

FIG. 3 is graphs each illustrating an example relationship between the vascular age and acceleration plethysmographic waveform (see Patent Literature 2 as well). FIG. 3(a) illustrates an example acceleration plethysmographic waveform obtained from a subject of low (young) vascular age. FIG. 3(b) in contrast illustrates an example acceleration pulse waveform obtained from a subject of high (old) vascular age. The subject of low vascular age specifically refers to an individual whose arteriosclerosis is not so much in progress. The subject of high vascular age in contrast refers to an individual whose arteriosclerosis is in progress to a certain extent.

It is known that an acceleration plethysmographic waveform varies depending on vascular age. FIG. 3(a) shows that for a young vascular age, Amplitude b is relatively large, and Amplitude d is relatively small. FIG. 3(b) in contrast shows that along with an increase in vascular age, Amplitude b becomes smaller, and Amplitude d becomes larger.

Accordingly, the vascular-age calculator 21 may calculate a waveform index (WI) indicated below and calculate the vascular age on the basis of the WI.

WI=d/a−b/a

A WI is an effective index indicating a vascular age. When calculating WIs in a plurality of skin regions, the vascular-age calculator 21 may calculate the vascular age by using a representative value (e.g., the average or central value) of these WIs.

Sex Detector 22

The sex detector 22 detects (determines) the sex of the subject H. By way of example, the sex detector 22 determines the sex of the subject H by analyzing an IMG. The sex detector 22 in the first embodiment identifies, through publicly known deep learning, whether the subject H appearing on the IMG is a male or a female. Information indicating the sex of the subject H (sex information) is another example of the foregoing attribute information. The sex detector 22 is thus another example of the foregoing attribute-information acquiring unit.

Attribute Classifier 23

The attribute classifier 23 classifies (detects) the attribute of the subject H (hereinafter, merely referred to as an attribute) on the basis of attribute information. An attribute in the Description refers to an attribute based on a vascular condition. To be specific, the attribute classifier 23 determines which pattern the attribute belongs to among predetermined patterns relating to a plurality of attributes. The total number of patterns is hereinafter expressed as N. N is any integer equal to or greater than two. In addition, each pattern is referred to as Pattern k. Herein, k is an integer that satisfies 1≤k≤N.

By way of example, the attribute classifier 23 may classify the attribute on the basis of the vascular age and sex of the subject H. For instance, the attribute classifier 23 may classify a plurality of subjects H in accordance with the age of their vascular ages and their sex. In this case, the attribute classifier 23 can classify the attribute of two different subjects (Subject HA and Subject HB) into different patterns. The attribute of each subject is listed below.
Subject HA is a male whose vascular age is in his 20 s.
Subject HB is a female whose vascular age is in her 30 s.

How the attribute classifier 23 classifies the attribute is not limited to the forgoing example. For instance, the attribute classifier 23 may classify the attribute on the basis of the amount of a waveform characteristic calculated by the pulse-wave-parameter calculator 20 (an example result of pulse wave analysis performed by the pulse-wave-parameter calculator 20).

The pulse-wave-parameter calculator 20 in the present example is used as the foregoing attribute-information acquiring unit. That is, information indicating the amount of a waveform characteristic (information about a waveform characteristic amount) can be used as the attribute information as well. It should be noted that the attribute information is not limited to vascular-age information and sex information.

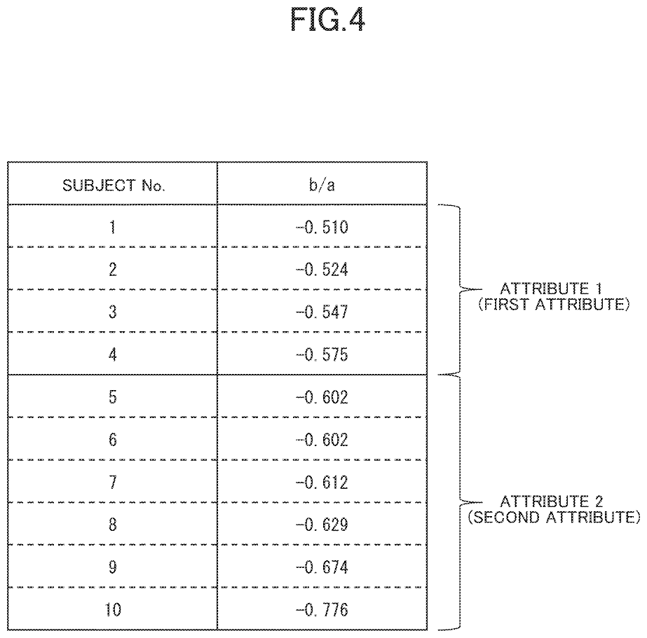

By way of example, the inventors of the present application (hereinafter, merely referred to as the inventors) used the pulse-wave-parameter calculator 20 to calculate the amplitude ratio b/a of an acceleration plethysmographic waveform as the amount of a waveform characteristic. The inventors then used the attribute classifier 23 to classify 10 subjects on the basis of the calculated amount. FIG. 4 is a table illustrating an example result of this classification. In the table of FIG. 4, the ratio of amplitude in each subject is listed in descending order.

In the example of FIG. 4, the attribute classifier 23 classifies the individual subjects into two groups by comparing the ratio of amplitude in each subject with a predetermined threshold. The threshold is set to −0.600 in the example of FIG. 4. To be specific, the attribute classifier 23 classifies the attribute of a certain subject as Attribute 1 when the ratio of amplitude in this subject is equal to or greater than the threshold. In contrast, the attribute classifier 23 classifies the attribute of another certain subject as Attribute 2 when the ratio of amplitude in this subject is less than the threshold. Attributes 1 and 2 are respectively examples of Patterns 1 and 2. It is noted that Attribute k is also referred to as a kth attribute.

Subsequently, the inventors created, with a method described later on, estimation models based on respective Attributes 1 and 2 (for convenience, referred to as attribute-specific models). Each estimation model is a calculation model for calculating (estimating) a blood pressure.

The inventors also created a conventional estimation model for comparison with the attribute-specific models. The conventional estimation model is different from the attribute-specific models in that it is not created for each attribute (i.e., the conventional model is common to all attributes). The conventional estimation model is hence also referred to as a common model.

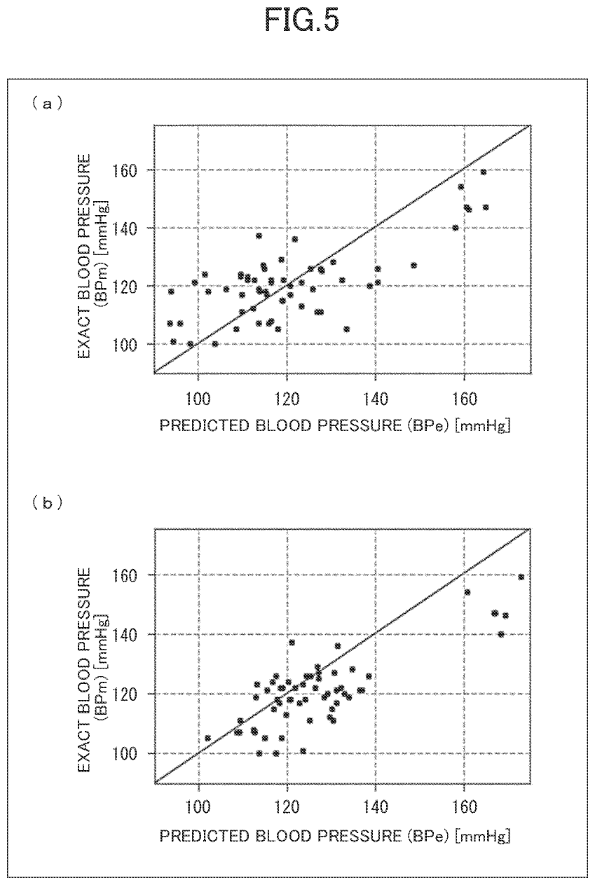

The inventors made a performance comparison between the common model and attribute-specific model. FIG. 5 illustrates an example result of the performance comparison. FIG. 5(a) is a graph illustrating an example relationship between exact blood pressure and predicted blood pressure in the common model. FIG. 5(b) is a graph illustrating an example relationship between exact blood pressure and predicted blood pressure in the attribute-specific model.

The exact blood pressures in FIG. 5 are blood pressures (i.e., BPms) measured by a cuff blood-pressure gauge (the blood-pressure acquiring unit 2). The BPms in FIG. 5 are example measurements (exact values) of blood pressure. In contrast, the predicted blood pressures in FIG. 5 are blood pressures (i.e., BPes) calculated by the model evaluating unit 40 (described later on) using a measurement model. The BPes in FIG. 5 are example predicted values of blood pressure.

The inventors calculated, using the model evaluating unit 40, a standard deviation (i.e., the standard deviation of error) between the exact blood pressure and predicted blood pressure for each of the common model and attribute-specific model. Referring to the common model, the calculation offered a standard deviation of 12.23 mmHg Referring to the attribute-specific model in contrast, the calculation offered a standard deviation of 9.25 mmHg.

The inventors also calculated, using the model evaluating unit 40, a mean square error (MSE) between the exact blood pressure and predicted blood pressure for each of the common model and attribute-specific model. Referring to the common model, the calculation offered an MSE of 149.64 mmHg Referring to the attribute-specific model in contrast, the calculation offered an MSE of 122.04 mmHg.

The foregoing experiment has revealed that the attribute-specific model can reduce an error better than the common model. As such, the attribute-specific model can improve the accuracy of blood pressure measurement better than the common model.

Supplemental Note N is not limited to 2 in the example of FIG. 4 as well. Let the total number of target subjects for classification be expressed as NT. Accordingly, N needs to be any natural number that satisfies 2≤N≤NT−1. Since NT=10 is satisfied in the example of FIG. 4, 2≤N≤9 is established. In this case, an N−1 number of thresholds including first to N−1th thresholds are set with regard to the ratio of amplitude, thus enabling an NT number of subjects to be classified into an N number of different patterns (first to Nth attributes). By way of example, these thresholds need to be set in the order such that the first threshold is the largest, the second threshold is the second largest, . . . , and the N−1th threshold is the smallest.

Model Creating Unit 30

The model creating unit 30 creates an estimation model. To be specific, the model creating unit 30 creates the estimation model by using, as training (learning) data, (i) the blood pressure (BPm) of the subject H acquired by the blood-pressure acquiring unit 2 and (i) a pulse wave parameter calculated by the pulse-wave-parameter calculator 20. The pulse wave parameter may be at least one of a PTT and the amount of a waveform characteristic.

As illustrated in FIG. 1, the model creating unit 30 has a first-model creating unit 300-1, a second-model creating unit 300-2, . . . , and an Nth-model creating unit 300-N. The kth-model creating unit 300-k creates an estimation model corresponding to the kth attribute (Pattern k). Herein, k is an integer that satisfies 1≤k≤N. As such, the model creating unit 30 can create an estimation model corresponding to each attribute.

In the Description, the first-model creating unit 300-1 to the Nth-model creating unit 300-N are also generically referred to as the model creating unit 30 for convenience. The description about the model creating unit 30 is applied to any kth-model creating unit 300-k. Likewise, in the Description, a first-model evaluation predicted-blood-pressure calculator 410-1 to an Nth-model evaluation predicted-blood-pressure calculator 410-N, all described later on, are also generically referred to as an evaluation predicted-blood-pressure calculator 41. In addition, a first-model evaluation-index calculator 420-1 to an Nth-model evaluation-index calculator 420-N, all described later on, are also generically referred to as the model evaluation-index calculator 42. Likewise, a first-model selector 600-1 to an Nth-model selector 600-N, all described later on, are also generically referred to as the model selector 60.

Example Method for Creating Estimation Model

Velocity v, the speed at which a pulse wave propagates through a blood vessel, is expressed by the Moens-Korteg equation as follows.

$$\begin{matrix}[ Expression 1 ] & \\ v = \sqrt{\frac{Ea}{2 R ρ}} & ( 1 )\end{matrix}$$

In Expression 1, E denotes the Young's modulus of the blood vessel, a denotes the wall pressure of the blood vessel, R denotes the diameter of the blood vessel, and ρ denotes blood density.

It is known that the Young's modulus E of a blood vessel exponentially varies with respect to blood pressure P. Accordingly, E is expressed as the follows, where E0 denotes the Young's modulus of a blood vessel in P=0.

[Expression 2]

E=E₀e^{γP} (2)

Here, γ denotes a constant that depends on the blood vessel.

The length L of a blood vessel pathway is expressed as follow.

[Expression 3]

L=vT (3)

Here, T denotes a pulse transit time (PTT), and L denotes the length of the blood vessel pathway.

Accordingly, Expression 4 is derived from Expressions 1 to 3, as indicated below.

$$\begin{matrix}[ Expression 4 ] & \\ P = \frac{1}{γ} ( ln \frac{1}{{T}^{2}} + ln \frac{2 R ρ {L}^{2}}{{E}_{0} a} ) & ( 4 )\end{matrix}$$

As shown in Expression 4, T and P establish a correlation when L is constant.

The model creating unit 30 may thus create at least one estimation model for a P by using a PTT (an example pulse wave parameter) calculated by the pulse-wave-parameter calculator 20.

For easy understanding, the following description addresses using only a PTT as the pulse wave parameter. However, the estimation model can be also created by using only the amount of a waveform characteristic instead of a PTT, as earlier described. Alternatively, the estimation model can be also created by using both a PTT and the amount of a waveform characteristic.

Firstly, the model creating unit 30 creates an estimation model M1 of Complexity Level 1. In the Description, a complexity level refers to the number of explanatory variables in an estimation model (e.g., the number of pulse wave parameters used in an estimation model). In the estimation model M1, a single PTT is used as an explanatory variable in the following instance.

In the following description, a single PTT calculated by the pulse-wave-parameter calculator 20 is expressed as a PTT1. The PTT1 is a PTT between any two skin regions. The model creating unit 30 performs regression analysis on the PTT1 and BPm through the method of least squares. The model creating unit 30 creates the estimation model M1 as a result of the regression analysis. Each PTT calculated by the pulse-wave-parameter calculator 20 and the BPm acquired by the blood-pressure acquiring unit 2 are both example training (learning) data.

Reference is made to an instance where the estimation model M1 is a linear model expressed by Expression 5 (a calculation model expressed by a linear function) below.

BP1=α1×PTT1+α2 (5)

In Expression 5, BP1 denotes a predicted blood pressure, and each of α1 and α2 is a constant. The model creating unit 30 in this case performs regression analysis to calculate α1 and α2 (that is, the model creating unit 30 creates the estimation model M1).

Hereinafter, the respective 1326 different PTTs in the example of FIG. 2 are referred to as PTT1-1 to PTT1-1326 for convenience. The model creating unit 30 uses PTT1-1 to PTT1-1326 to create as many estimation models M1 as these PTTs. The respective estimation models M1 are referred to as M1-1 to M1-1326 for convenience.

Secondly, the model creating unit 30 creates an estimation model M2 of Complexity Level 2. In the estimation model M2, two PTTs are used as explanatory variables. In the following description, two mutually different PTTs calculated by the pulse-wave-parameter calculator 20 are expressed as PTT1 and PTT2.

The model creating unit 30 performs regression analysis on (i) the PTT1 and PTT2 and (ii) the BPm through the method of least squares. The model creating unit 30 creates the estimation model M2 as a result of the regression analysis.

Reference is made to an instance where the estimation model M2 is a linear model expressed by Expression 6 below.

BP2=β1×PTT1+β2×PTT2+β3 (6)

In Expression 6, BP2 denotes a predicted blood pressure, and each of β1 to β3 is a constant. The model creating unit 30 in this case performs regression analysis to calculate β1 to β3.

In the example of FIG. 2, the model creating unit 30 uses PTT1-1 to PTT1-1326 to create more estimation models M2 than these PTTs. There are 878475 combinations (i.e., ₁₃₂₆C₂ combinations) of the PTT1 and PTT2 in this example. The model creating unit 30 thus creates 878475 estimation models M2.

As earlier described, the estimation model M2 can be created by using both a PTT and the amount of a waveform characteristic. In this case for instance, the estimation model M2 may be created by using the PTT as a first explanatory variable and by using the amount of a waveform characteristic as a second explanatory variable.

Likewise, the model creating unit 30 creates an estimation model M3 of Complexity Level 3, an estimation model M4 of Complexity Level 4 . . . , and an estimation model Mz of Complexity Level z. Herein, z denotes the maximum of the complexity level. In addition, z may be set, as appropriate, by the manufacturer of the model setting device 100. The model creating unit 30 supplies each created estimation model to the model evaluating unit 40 (more specifically, to the evaluation predicted-blood-pressure calculator 41).

As described above, the first-model creating unit 300-1 creates an estimation model corresponding to on Attribute 1 (hereinafter, a first model). Likewise, the second-model creating unit 300-2 to the Nth-model creating unit 300-N creates their estimation models. That is, the kth-model creating unit 300-k creates an estimation model corresponding to Attribute k (hereinafter, a kth model).

The model creating unit 30 creates the first to Nth models in this way. The first to Nth models are hereinafter also generically referred to as an estimation model group. In the example of FIG. 1, the model creating unit 30 supplies the created estimation model group to the model evaluating unit 40 and model storage 55.

Model Evaluating Unit 40

The model evaluating unit 40 individually evaluates estimation models created by the model creating unit 30 and outputs the results of their evaluation. To be specific, the model evaluating unit 40 outputs a PI, described below, as the result of the evaluation. In the example of FIG. 1, the model evaluating unit 40 acquires an estimation model group directly from the model creating unit 30. In some embodiments, the model evaluating unit 40 may acquire an estimation model group stored in advance in the model storage 55.

The model evaluating unit 40 has the evaluation predicted-blood-pressure calculator 41 and the model evaluation-index calculator 42. The evaluation predicted-blood-pressure calculator 41 has the first-model evaluation predicted-blood-pressure calculator 410-1, the second-model evaluation predicted-blood-pressure calculator 410-2 . . . , and the Nth-model evaluation predicted-blood-pressure calculator 410-N. The model evaluation-index calculator 42 has the first-model evaluation-index calculator 420-1, the second-model evaluation-index calculator 420-2 . . . , and the Nth-model evaluation-index calculator 420-N.

The kth-model evaluation predicted-blood-pressure calculator 410-k and the kth-model evaluation-index calculator 420-k are functional units appropriate to the kth model. The kth-model evaluation predicted-blood-pressure calculator 410-k and the kth-model evaluation-index calculator 420-k are also generically referred to as a kth-model evaluating unit.

The evaluation predicted-blood-pressure calculator 41 calculates a predicted blood pressure (hereinafter, BPe) in an estimation model created by the model creating unit 30. To be specific, the evaluation predicted-blood-pressure calculator 41 calculates the BPe by applying (to be specific, substituting), to the estimation model, a pulse wave parameter calculated as test data by the pulse-wave-parameter calculator 20.

The model evaluation-index calculator 42 calculates the evaluation index (hereinafter, PI) of the estimation model. The model evaluation-index calculator 42 may calculate the PI on the basis of the BPm and BPe. By way of example, the model evaluation-index calculator 42 calculates, as the PI, an MSE between the BPm and BPe. The model evaluation-index calculator 42 calculates the PI of each estimation model in ascending order of the complexity levels of the estimation models. The model evaluation-index calculator 42 then supplies the calculated PI to the model storage 55.

The PI (evaluation index) is not limited to an MSE. The PI is any index that can be calculated on the basis of a BPm and a Bpe (see a second embodiment described later on). By way of example, the average of error (e.g., mean absolute error) between the BPm and BPe may be used as the PI. Alternatively, the standard deviation of error between the BPm and BPe may be used as the PI.

Alternatively, a plurality of predetermined parameters (numeric values) may be calculated based on the BPm and BPe, and these parameters may undergo ranking (e.g., the parameters may undergo determination on which is superior and which is inferior). In this case, a numeral indicating the rank of each parameter may be used as the PI.

As described above, the first-model evaluation predicted-blood-pressure calculator 410-1 calculates a predicted blood pressure for the first model (a first-model predicted blood pressure). Likewise, the second-model evaluation predicted-blood-pressure calculator 410-2 to the Nth-model evaluation predicted-blood-pressure calculator 410-N calculate their predicted blood pressures. That is, the kth-model evaluation predicted-blood-pressure calculator 410-k calculates a predicted blood pressure for a kth-model (hereinafter, BPek). The evaluation predicted-blood-pressure calculator 41 calculates BPe1 to BPeN in this way. Hereinafter, BPe1 to BPeN are also generically referred to as a group of predicted blood pressures. The evaluation predicted-blood-pressure calculator 41 supplies the calculated group of predicted blood pressures to the model evaluation-index calculator 42.

Subsequently, the first-model evaluation-index calculator 420-1 calculates each PI of the first model (a set of first-model evaluation indexes). Likewise, the second-model evaluation-index calculator 420-2 to the Nth-model evaluation-index calculator 420-N calculate their sets of model evaluation indexes. That is, the kth-model evaluation-index calculator 420-k calculates a set of kth-model evaluation indexes (hereinafter, PIk).

Hereinafter, PI1 to PIk are also generically referred to as a group of evaluation index sets. The model evaluation-index calculator 42 associates the calculated group of evaluation index sets with the estimation model group and then supplies the group of evaluation index sets to the model storage 55.

Measurement Model Setting Performed by Model Evaluating Unit 40

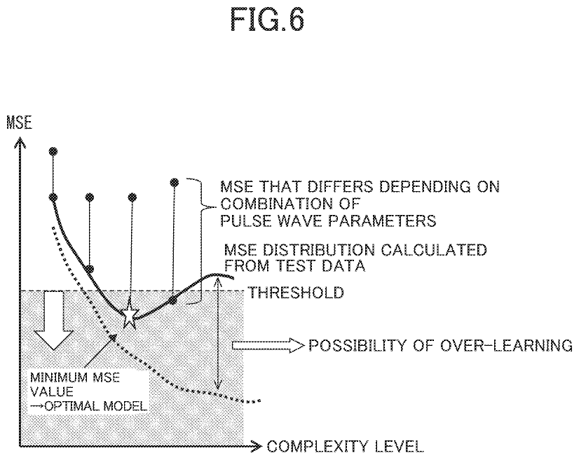

The model evaluating unit 40 sets (selects) at least one measurement model on the basis of the results (each PI) of evaluation made by the model evaluating unit 40 (more specifically, the model evaluation-index calculator 42). To be specific, the model evaluating unit 40 selects at least one measurement model from among one or more estimation models stored in the model storage 55. With reference to FIG. 6, the following describes an instance where a single measurement model is selected. FIG. 6 illustrates an example process for the model evaluating unit to set a measurement model.

As illustrated in FIG. 6, the model evaluating unit 40 selects, as a measurement model, an estimation model that has a minimum MSE from among plotted blood-pressure-estimation models having the smallest MSE (an example PI) in each complexity level. In the example of FIG. 6, a single predetermined estimation model of Complexity Level 3 is selected as the measurement model (see the star-shaped legend in FIG. 6).

In the blood-pressure measuring device 1, it is preferable that data (training data) for the model creating unit 30 to create a blood-pressure-estimation model be different from data (test data) for the model evaluating unit 40 to estimate the blood-pressure-estimation model. The model evaluating unit 40 in this case can select a measurement model that is well applicable to test data and has a high ability of generalization without over-learning.

How to set a measurement model is not limited to the foregoing example. For instance, the model evaluating unit 40 may extract, as measurement model candidates, estimation models each of which has a PI (e.g., an MSE) equal to or less than a predetermined threshold from among one or more estimation models. The model evaluating unit 40 may then select at least one measurement model from among the measurement model candidates. By way of example, the model evaluating unit 40 may select, as the measurement model, an estimation model having a PI that is the smallest of those of the measurement model candidates. Alternatively, the model evaluating unit 40 may select, as the measurement model, an estimation model having a complexity level that is the smallest of those of the measurement model candidates.

As described above, the model evaluating unit 40 selects at least one measurement model (intra-first-model measurement model) from among one or more first models. This also holds true for the second to Nth models. That is, the model evaluating unit 40 specifies at least one measurement model (intra-kth-model measurement model) from among one or more kth models. The intra-kth-model measurement model is a calculation model for the blood-pressure measuring unit 160 to measure the subject's blood pressure (P) when he/she falls under Attribute k.

When the kth-model creating unit 300-k creates only one kth model, the model evaluating unit 40 needs to select this kth model as the measurement model (intra-kth-model measurement model). Hereinafter, the intra-first-model measurement model to the intra-Nth-model measurement model are also generically referred to as a measurement model group. The model evaluating unit 40 supplies the selected measurement model group to the model storage 55.

Model Storage 55

The model storage 55 may be a publicly known storing device that can store (retain) individual data pieces. In the first embodiment, the model storage 55 stores (i) an estimation model group created by the model creating unit 30. It is preferable that the model storage 55 also store (i) a group of evaluation index sets calculated by the model evaluation-index calculator 42 and (ii) a measurement model group selected by the model evaluating unit 40.

How to Set Measurement Model

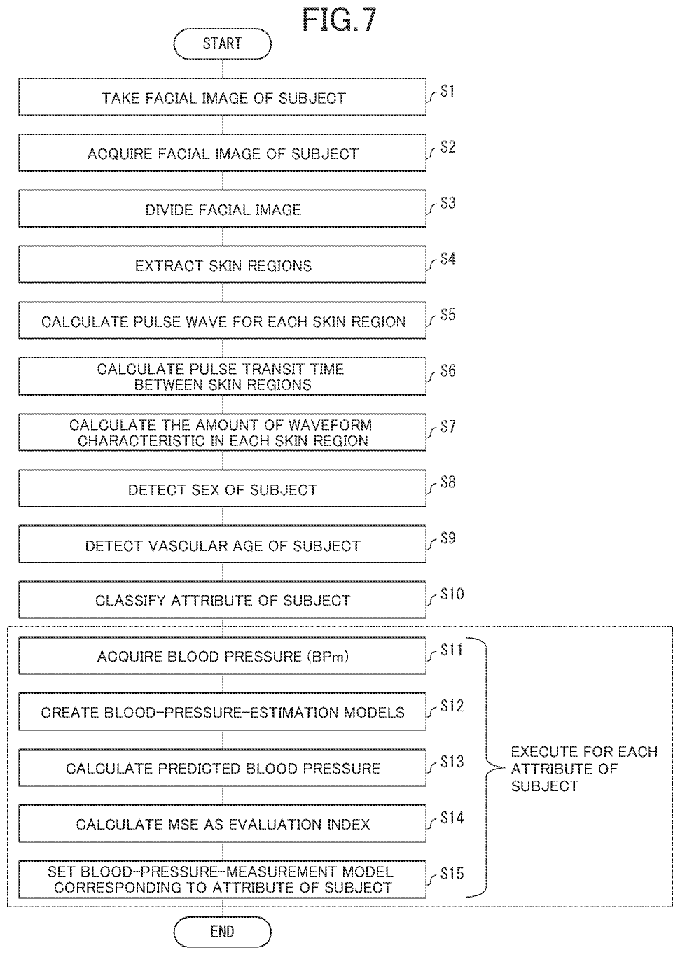

FIG. 7 is a flowchart illustrating an example process performed by the model setting device 100. FIG. 7 illustrates, by way of example, how the model setting device 100 sets a measurement model (an example method for setting a measurement model).

The first step is S1, where the image pickup unit 11 captures a subject image. In S2, the facial-image acquiring unit 14 acquires a facial image (IMG) from the captured subject image. In S3, the facial-image divider 15 divides the IMG into a plurality of partial regions. In S4, the skin region extractor 16 extracts skin regions from among the partial regions. In S5, the pulse wave calculator 17 calculates a pulse wave (pulse wave signal) for each skin region. S1 to S5 are also generically referred to as a step of pulse wave acquisition.

Subsequently, the pulse-wave-parameter calculator 20 calculates a pulse wave parameter on the basis of the pulse waves. Firstly, in S6, the pulse-wave-parameter calculator 20 calculates a pulse transit time (PTT) between the skin regions. In S7, the pulse-wave-parameter calculator 20 then calculates the amount of a waveform characteristic in each skin region. S6 and S7 are also generically referred to as a step of calculating a pulse wave parameter.

The next step is S8, where the sex detector 22 detects the sex of a subject H by analyzing the IMG. In S9, the vascular-age calculator 21 calculates the vascular age of the subject H on the basis of the pulse waves. S8 and S9 are also generically referred to as a step of attribute information acquisition.

In S10, i.e., a step of attribute classification, the attribute classifier 23 then classifies the attribute of the subject H on the basis of the attribute information. In this example, the attribute classifier 23 classifies the attribute of the subject H on the basis of the foregoing vascular-age information and sex information. For instance, the attribute classifier 23 classifies the attribute of the subject H into any one of Attributes 1 to N (Attribute k).

The following individual process steps are performed for each Attribute k. Firstly, in S11, i.e., a step of acquiring a second blood pressure, the blood-pressure acquiring unit 2 acquires the blood pressure (BPm, i.e., the second blood pressure) of the subject H.

Then, the model creating unit 30 (more specifically, the kth-model creating unit 300-k) uses this training data to create one or more estimation models (kth models) for Attribute k. To be specific, the model creating unit 30 uses the pulse wave parameter and BPm to create each estimation model. This process step is S12, i.e., a step of model creation. It is noted that the BPm used in S12 is a blood pressure measured by the blood-pressure acquiring unit 2 at the same time as the capturing (S1) of the subject image. That is, the step of measuring the second blood pressure is executed once in advance at the same time as S1 prior to S11.

Next, the evaluation predicted-blood-pressure calculator 41 (more specifically, the kth-model evaluation predicted-blood-pressure calculator 410-k) calculates, using the test data, a predicted blood pressure (BPe, more specifically, BPek) in each estimation model. To be specific, in S13, the evaluation predicted-blood-pressure calculator 41 calculates the BPes by applying the pulse wave parameter to each estimation model.

Next, the model evaluation-index calculator 42 (more specifically, the kth-model evaluation-index calculator 420-k) calculates the evaluation index (PI, more specifically, PIk) of each estimation model. To be specific, in S14, the model evaluation-index calculator 42 calculates a mean square error (MSE) between the BPe and BPm as the PI. S13 and S14 are also generically referred to as a step of model evaluation.

The model evaluating unit 40 then selects at least one measurement model (intra-kth-model measurement model) from among the estimation models on the basis of the results (PIs) of the evaluation made by the model evaluating unit 40. For instance, the model evaluating unit 40 sets, as the intra-kth-model measurement model, a model having an MSE that is the smallest of those of the kth models. This process step is S15, i.e., a step of model setting. S11 to S15 are performed on each of Classifications 1 to N, thus enabling the model setting device 100 to set the intra-first-model measurement model to the intra-Nth-model measurement model.

Pulse-Wave-Signal Quality Evaluating Unit 150

The following describes the remaining functional unit of the blood-pressure measuring device 1. The pulse-wave-signal quality evaluating unit 150 evaluates the quality of a pulse wave signal in each skin region, which is used for the blood-pressure measuring device 1 (more specifically, the blood-pressure measuring unit 160) to measure a blood pressure (P). By way of example, the pulse-wave-signal quality evaluating unit 150 calculates the SNR of a pulse wave signal as an index indicating the quality of the pulse wave signal.

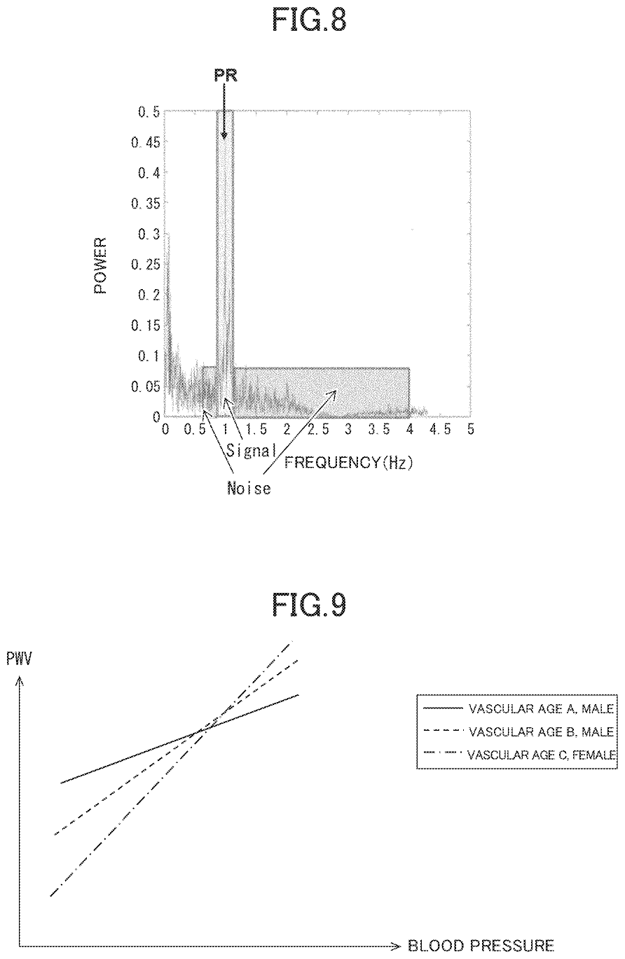

FIG. 8 is a graph illustrating an example power spectrum of the pulse wave signal (hereinafter, merely referred to as a power spectrum). The lateral axis of the graph indicates frequency, and the lateral axis of the same indicates the power of the pulse wave signal. The pulse-wave-signal quality evaluating unit 150 performs frequency analysis on the pulse wave signal to derive the power spectrum.

Pulse waves propagate to arteries by heart's pumping action. A pulse wave signal hence has a constant period according to heartbeat. In many cases, the peak of a power spectrum is found in a frequency band of around 1 Hz when the subject H remains at rest. PR (pulse rate) in FIG. 8 is an example of the peak.

Accordingly, the pulse-wave-signal quality evaluating unit 150 may calculate a signal component (Signal) and a noise component (Noise) within a predetermined bandwidth. By way of example, the pulse-wave-signal quality evaluating unit 150 may set a frequency band of ±0.05 Hz centering on PR as a signal band. The pulse-wave-signal quality evaluating unit 150 then calculates the total power of the power spectrum within the signal band as Signal. On the other hand, the pulse-wave-signal quality evaluating unit 150 may set, as a noise band, a frequency band ranging from 0.75 to 4.0 Hz and excluding the Signal band. The pulse-wave-signal quality evaluating unit 150 then calculates the total power of the power spectrum within the noise band as Noise. The pulse-wave-signal quality evaluating unit 150 then calculates an SNR, which is a signal-to-noise ratio.

In some cases, a pulse wave of predetermined signal quality (high-accuracy pulse wave) cannot be obtained from some of the skin regions. Examples of such skin regions include (i) a skin region partly covered with an object and (ii) a skin region with a shade casted thereon. To improve the accuracy of blood pressure measurement, the fact that there are such skin regions is preferably reflected by using a resultant quality evaluation on a pulse wave signal made by the pulse-wave-signal quality evaluating unit 150.

By way of example, the pulse-wave-signal quality evaluating unit 150 may classify the individual skin regions into (i) a region where a pulse wave of predetermined signal quality has been obtained (hereinafter, a quality-compliant region) and (ii) the other region (hereinafter, a quality-noncompliant region). The quality-noncompliant region can be also expressed as a region where a pulse wave of predetermined signal quality has not been obtained. Reference is made to an instance where such predetermined signal quality can be expressed as SNR >0.15. The pulse-wave-signal quality evaluating unit 150 in this case identifies a region that satisfies SNR >0.15 as a quality-compliant region from among the skin regions.

Model Selector 60

The model selector 60 has the first-model selector 600-1, the second-model selector 600-2 . . . , and the Nth-model selector 600-N. The kth-model selector 600-k is a functional unit appropriate to the kth model. The model selector 60 operates for the blood-pressure measuring unit 160 to measure a blood pressure (P) after the model setting device 100 finishes processing.

The model selector 60 reads a measurement model group previously set by the model setting device 100 (the model evaluating unit 40) from the model storage 55. The model selector 60 then selects, from the measurement model group, a measurement model corresponding to the attribute of the subject H classified by the attribute classifier 23. That is, the model selector 60 selects a measurement model corresponding to Attribute k (Pattern k). The measurement model selected by the model selector 60 is at least one measurement model that is used for the blood-pressure measuring unit 160 to measure a blood pressure (a model for blood pressure measurement). The following describes an instance where there is a single model for blood pressure measurement.

To be specific, the first-model selector 600-1 selects a single measurement model (a first model for blood pressure measurement) from among one or more measurement models corresponding to Attribute 1 (intra-first-model measurement models). This holds true for the second-model selector 600-2 to the Nth-model selector 600-N. That is, the kth-model selector 600-k selects a single measurement model (a kth model for blood pressure measurement) from among one or more intra-kth-model measurement models.

As described above, the model selector 60 selects a measurement model from among one or more blood-pressure-estimation models on the basis of the resultant evaluations (PIs) of these respective estimation models.

When there is only one intra-kth-model measurement model created in the model evaluating unit 40, the kth-model selector 600-k needs to select this single intra-kth-model measurement model as a kth model for blood pressure measurement. It should be thus noted that the pulse-wave-signal quality evaluating unit 150 is not an essential component of the blood-pressure measuring device 1.

However, to improve the accuracy of a resultant measurement (P) made by the blood-pressure measuring device 1, the pulse-wave-signal quality evaluating unit 150 is preferably provided. For instance, the model selector 60 may select a measurement model for blood pressure measurement on the basis of a resultant quality evaluation of a pulse wave signal made by the pulse-wave-signal quality evaluating unit 150. For instance, the kth-model selector 600-k may select, as a kth model for blood pressure measurement, a model having a pulse wave signal of the highest quality of one or more intra-kth-model measurement models.

The model selector 60 may also extract, as candidates for a model for blood pressure measurement, only models that use only a quality-compliant region from the measurement model group. This can avoid reduction in the accuracy of measurement in the blood-pressure measuring device 1 with more certainty.

Blood-Pressure Measuring Unit 160 and Blood-Pressure-Measurement Outputting Unit 170

The blood-pressure measuring unit 160 measures a blood pressure (P) on the basis of a pulse wave parameter by using an estimation model (kth model) corresponding to Attribute k. More specifically, the blood-pressure measuring unit 160 measures the P by using a model for blood pressure measurement selected by the model selector 60. That is, the blood-pressure measuring unit 160 calculates the P by applying the pulse wave parameter, calculated by the pulse-wave-parameter calculator 20, to the model for blood pressure measurement. In this way, the blood-pressure measuring unit 160 calculates the P on the basis of the pulse wave parameter by using the model for blood pressure measurement.

As described above, the model selector 60 selects a model for blood pressure measurement corresponding to Attribute k (kth model for blood pressure measurement). The blood-pressure measuring unit 160 can thus calculate the P by using a model for blood pressure measurement that is suitable for the attribute of the subject H.

The blood-pressure-measurement outputting unit 170 acquires a P measured by the blood-pressure measuring unit 160. The blood-pressure-measurement outputting unit 170 then outputs the P as a resultant blood pressure measurement. The blood-pressure-measurement outputting unit 170 may output the P through any notification. By way of example, the blood-pressure-measurement outputting unit 170 may be a display. The blood-pressure-measurement outputting unit 170 in this case can visually provide the subject H with the resultant blood pressure measurement by displaying a numeric value indicating the P.

The blood-pressure-measurement outputting unit 170 may display at least some of various attribute information pieces (e.g., vascular-age information) along with the numeric number indicating the P. In this case, the fact that blood pressure measurement based on the attribute of the subject H is ongoing can be suggested to the subject H.

How to Measure Blood Pressure

The following describes, by way of example, how the blood-pressure measuring device 1 measures a blood pressure (a blood-pressure measurement method). Individual process steps included in the blood-pressure measurement method are executed after all the process steps in FIG. 7 complete. Thus in the following instance, the model storage 55 stores an estimation model group, a group of evaluation index sets, and a measurement model group, all derived by the model setting device 100, in advance prior to the start of each process step in the blood-pressure measurement method.

Firstly, the blood-pressure measurement method includes process steps similar to S1 to S10 in FIG. 7. That is, the blood-pressure measurement method includes, like the model setting method, a step of pulse wave acquisition, a step of calculating a pulse wave parameter, a step of attribute information acquisition, and a step of attribute classification.

Thereafter, a step of measuring a first blood pressure is performed, where the blood-pressure measuring unit 160 calculates a P on the basis of the pulse wave parameter by using the kth model, as earlier described. More specifically, a step of model selection is performed, where the model selector 60 selects a measurement model corresponding to Attribute k (more strictly, a kth model for blood pressure measurement) prior to the step of measuring the first blood pressure. The step of measuring the first blood pressure is then performed, where the blood-pressure measuring unit 160 uses the measurement model to calculate the P.

Effects

Unlike a contact blood-pressure measuring device (e.g., the blood-pressure acquiring unit 2, which is a cuff blood-pressure gauge), a contactless blood-pressure measuring device (e.g., the blood-pressure measuring device 1) cannot acquire the blood pressure of the subject H directly as a physical quantity. A contactless blood-pressure measuring device hence needs to set a model for deriving his/her blood pressure (P) from the living body information about the subject H (e.g., a pulse wave parameter).

However, it is known that the correlation between blood pressure and pulse wave velocity (PWV for short, which is the velocity of pulse wave propagation) can differ significantly depending on elements, such as the age and sex of the subject H, as shown in FIG. 9. FIG. 9 is a graph illustrating an example relationship between blood pressure and PWV for each of subjects having different attributes. FIG. 9 shows an example relationship between blood pressure and PWV for each of the following subjects: a male of a certain vascular age (Vascular Age A); a male of another vascular age (Vascular Age B); and a female of another vascular age (Vascular Age C). It is noted that PWV correlates negatively with PTT. The PWV in FIG. 9 may be hence read as PTT.

As demonstrated in the graph, it is not necessarily true that a single model suitable for measuring the blood pressure of a certain subject (e.g., the male of Vascular Age A) is also suitable for measuring the blood pressure of another subject (e.g., the male of Vascular Age B or the female of Vascular Age C). Hence, the foregoing common model fails to sufficiently improve the accuracy of measurement of a P, because variations between individual subjects H (e.g., age and sex) cannot be reflected when this common model is used.

In view of the foregoing, the inventors have arrived at a new configuration, that is, “measuring a P by using an individual measurement model that is set based on the attribute of a subject H”. More essentially, the inventors have arrived at a new configuration, that is, “creating at least one kind of estimation model corresponding to the attribute of each subject H”. These new configurations enable blood pressure measurement reflecting variations between the individual subjects H, thereby further improving the accuracy of measurement of the P than before (see also FIG. 5).

Attribute classification based on the actual age of each subject H (hereinafter, merely referred to as actual age) is a possible way to create at least one kind of estimation model corresponding to the attribute of the subject H (see Patent Literature 1). However, an actual age seems to be not necessarily sufficient as an index indicating the vascular condition of the subject H. This is because that for instance, the vascular age of the subject H may be high even though his/her actual age is low (and vice versa), depending on his/her lifestyle (e.g., eating habit and fitness habit).

In view of this regard, it is preferable that instead of the actual age of the subject H, his/her vascular age be used as an index indicating his/her vascular condition in order to improve the measurement accuracy of the P. Accordingly, the example process in FIG. 7 includes attribute classification based on the vascular age. Such attribute classification can create an estimation model that more conforms to the actual vascular condition of the subject H than attribute classification based on the actual age.

The model setting device 100 can acquire attribute information through, for instance, IMG analysis. This eliminates the need for a user of the blood-pressure measuring device 1 to manually select a measurement model suitable for the subject H. This also eliminates the need to ask the subject H about his/her attribute information and to get the answer to the attribute information directly from him/her. As such, the blood-pressure measuring device 1 can measure the P more easily and more accurately than before.

Supplemental Note 1

As described in Patent Literature 3, it is known that the apparent age of the subject H (hereinafter, referred to as apparent age) has a strong correlation with his/her vascular age. For instance, enlarged blemishes, increased pores, and cheek sagging become conspicuous along with the aging of blood vessels. Apparent age hence tends to be higher as vascular age increases.

In the first embodiment, the process may include attribute classification based on apparent age instead of vascular age. The attribute-information acquiring unit (e.g., the vascular-age calculator 21) in this case needs to calculate an apparent age instead of a vascular age. This apparent-age calculation may use a publicly known method (see Patent Literature 4 for instance). For instance, the attribute-information acquiring unit calculates the apparent age through IMG analysis. In this way, the attribute information can include information indicating the apparent age (apparent-age information).

Vascular age and apparent age are also generically referred to as blood-vessel-related age. In addition, information indicating the blood-vessel-related age is also referred to as blood-vessel-related age information. Both vascular-age information and apparent-age information are examples of the blood-vessel-related age information.

Using blood-vessel-related age information as the attribute information can reflect the actual vascular condition of the subject H. Likewise, using information about the amount of a waveform characteristic as the attribute information can reflect the actual vascular condition of the subject H. Moreover, the attribute information desirably includes sex information. This enables the sex difference of the subject H to be further reflected.

Supplemental Note 2

As described above, the essential concept of the blood-pressure measuring device according to the aspect of the present disclosure lies in the following: classifying the subject H on the basis of his/her attribute information, and using an estimation model suitable for the subject H in accordance with the result of the classification. It should be thus noted that measurement model selection based on resultant evaluations of respective estimation models is not an indispensable process in the blood-pressure measuring device. That is, the selector can be omitted from the blood-pressure measuring device according to the aspect of the present disclosure.

As seen from the foregoing, the model storage does not necessarily have to store in advance a group of evaluation index sets and a measurement model group. The model evaluating unit can be thus omitted from the model setting device according to the aspect of the present disclosure.

Modifications

(1) Some medicines affect the vascular condition of the subject H considerably. The subject H, if taking these medicines, can hence have a facial swelling resulting from a change in his/her vascular condition. Accordingly, the attribute-information acquiring unit may perform IMG analysis to determine whether the subject H has a facial swelling.

That is, the attribute-information acquiring unit may perform IMG analysis to acquire information indicating whether the subject H has a facial swelling (swelling information) as his/her attribute information. This enables attribute classification based on whether the subject H has a facial swelling. In other words, attribute classification can be performed that reflects whether the subject H has taken any of the foregoing medicines. Such attribute classification has an effect similar to that in the first embodiment.

(2) As a matter of course, attribute classification can be also performed by combining the foregoing various different pieces of attribute information. By way of example, the attribute information according to the aspect of the present disclosure needs to include at least any of vascular-age information, apparent-age information, sex information, information about the amount of a waveform characteristic, and swelling information.

### Second Embodiment

The first embodiment has described performing attribute classification on a subject by using vascular-age information, calculated based on a pulse wave signal calculated by the pulse wave calculator 17, and by using sex information, detected based on a facial image acquired by the facial-image acquiring unit 14.

The second embodiment is an alternative to or modification of the first embodiment. The second embodiment provides the following: instead of vascular-age information calculated based on a pulse wave signal, measuring a blood pressure by using a cuff blood-pressure gauge or other things; classifying a subject on the basis of an average blood pressure that can be calculated from the resultant measurement (this average blood pressure can be calculated by diastolic blood pressure+systolic blood pressure×⅓); and model setting and blood pressure measurement according to the classification.

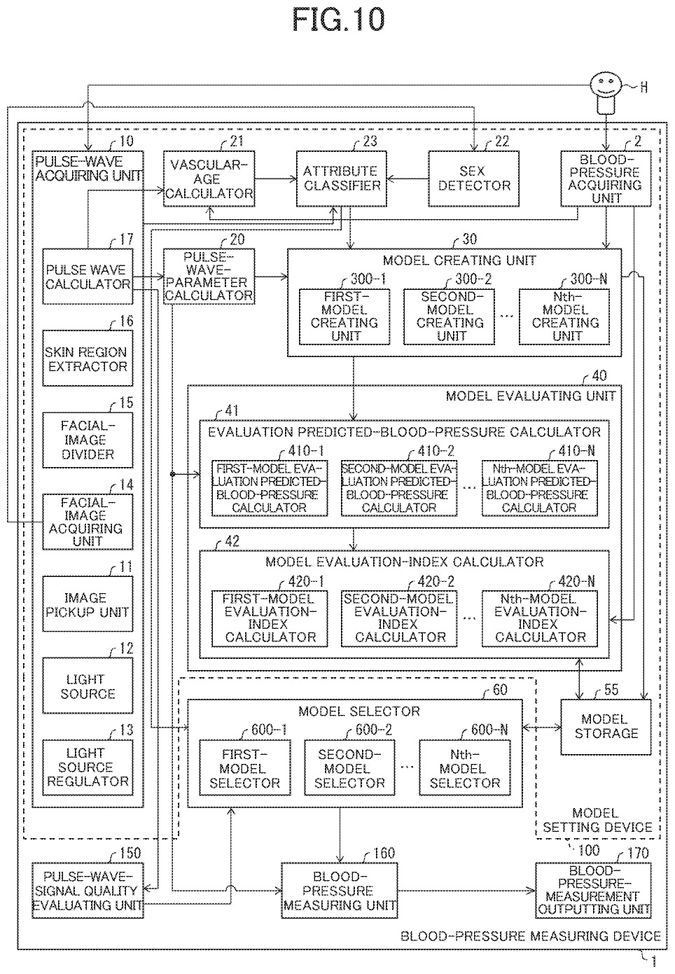

FIG. 10 is a functional block diagram illustrating the configuration of main components of the blood-pressure measuring device 1 according to the second embodiment. The following describes the blood-pressure acquiring unit 2, the vascular-age calculator 21, and the attribute classifier 23, all of which function in a manner different from that described in the first embodiment. The blood-pressure acquiring unit 2 outputs a BPm to the vascular-age calculator 21 as well as to the model creating unit 30 and model evaluating unit 40.

To select an optimal blood-pressure-estimation model for a subject H, the vascular-age calculator 21 calculates information about the vascular age of the subject H as attribute information of the subject H on the basis of the blood pressure of the subject H acquired by the blood-pressure acquiring unit 2. The vascular-age calculator 21 calculates an average blood pressure from the blood pressure value (a systolic blood pressure or SBP, and a diastolic blood pressure or DBP) acquired by the blood-pressure acquiring unit 2. The average blood pressure can be calculated by DBP+⅓*(SBP−DBP) for instance. The average blood pressure calculated by the vascular-age calculator 21 is output to the attribute classifier 23.

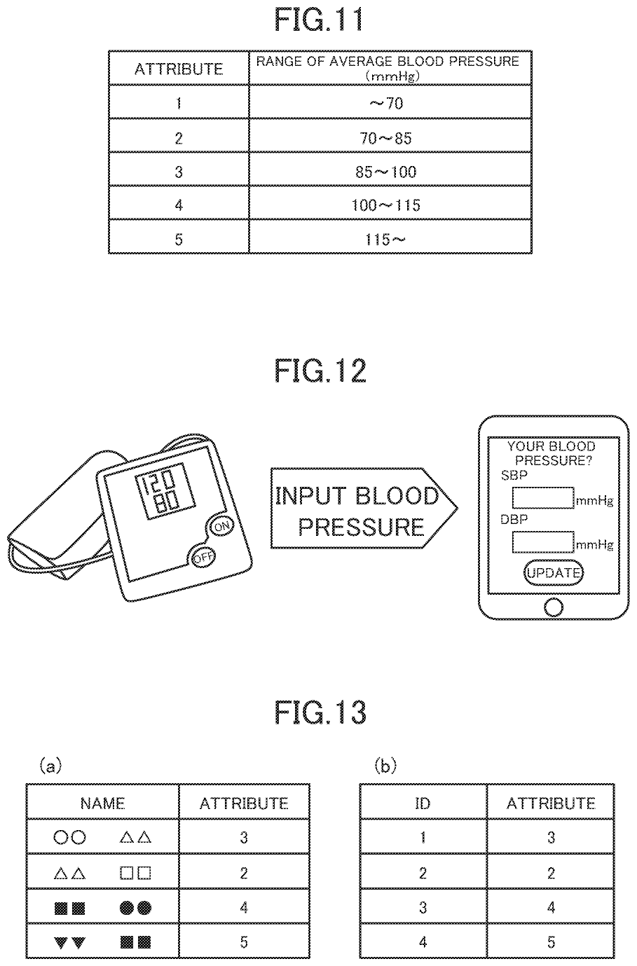

The attribute classifier 23 classifies the subject H on the basis of the average blood pressure, reflecting the vascular-age information calculated from the cuff blood pressure value, and on the basis of sex information, detected by the sex detector 22. FIG. 11 illustrates, by way of example, attribute classifications based on values of average blood pressure (classification indexes). In this case, the attribute classifier 23 determines which of the attributes the subject is classified into and inputs the resultant determination to the model selector 60.

It is noted that attribute classification does not need to be performed on the subject H every time his/her blood pressure is measured, but needs to be performed at least once before measurement. Nevertheless, periodic attribute classification (for instance, once in several months to several years) offers an updated model reflecting the latest vascular condition of the subject H.

The blood-pressure acquiring unit 2, the vascular-age calculator 21, and the attribute classifier 23 do not necessarily have to be disposed inside the blood-pressure measuring device 1. For blood pressure measurement using, as the blood-pressure measuring device 1, a camera installed in a smartphone, a blood pressure value measured by a contact blood-pressure gauge (e.g., a cuffblood-pressure gauge) separate from the body of the blood-pressure measuring device may be manually input, as illustrated in FIG. 12.

As illustrated in FIGS. 13(a) and (b), the blood-pressure measuring unit 160 associates information from which the subject H himself/herself, such as the name, ID and facial image (face recognition) of the subject H, can be identified, with the attribute of the subject H or with at least one estimation model corresponding to his/her attribute, and the blood-pressure measuring unit 160 also keeps the associated information. This configuration eliminates the need to perform model selection every time a blood pressure is measured, thereby enabling the blood-pressure measuring unit 160 to perform measurement even when the unit is not communicably connected to the model selector 60. In some embodiments, the model selector 60 does not have to be disposed inside the blood-pressure measuring device 1; for instance, the model selector 60 disposed outside the blood-pressure measuring device 1 may be communicably connected to the blood-pressure measuring device 1.

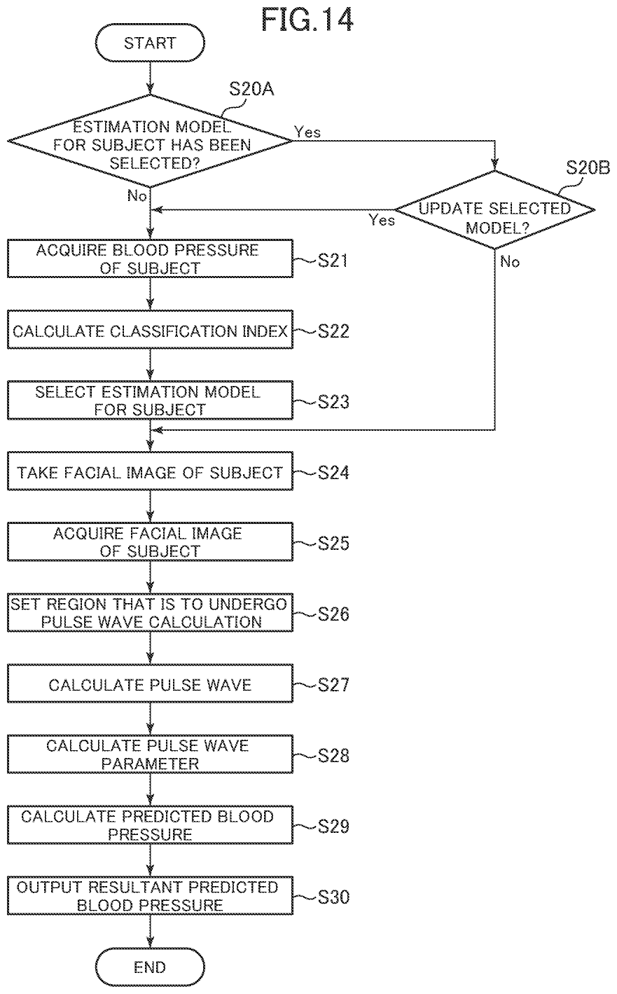

FIG. 14 is a flowchart illustrating an example process performed by the blood-pressure measuring device. FIG. 14 illustrates, by way of example, how the blood-pressure measuring device 1 measures a blood pressure.

As illustrated in FIG. 14, how the blood-pressure measuring device 1 performs blood pressure measurement includes the following process steps: S20A, where the blood-pressure measuring device 1 determines whether an estimation model for a target measurement subject has been selected; and S21 to S23, which are performed if such an estimation model for the target measurement subject has not been selected (if NO in S20A). If an estimation model for the target measurement subject has been selected (if YES in S20A), the process proceeds to S20B, where the blood-pressure measuring device 1 determines whether to update the selected model. For model update even when there is already a model selected (if YES in 520B), the process proceeds to S21 to S23 below. If the blood-pressure measuring device 1 determines not to update the selected model (if NO in 520B), the process does not proceed to S21 to S23 below but proceeds to S24 and the subsequent process steps.

In S21, the blood-pressure acquiring unit 2 measures the blood pressure of the target subject. Then in S22, the vascular-age calculator 21 calculates a classification index (average blood pressure) on the basis of the blood pressure acquired by the blood-pressure acquiring unit. Then in S23, the attribute classifier determines which attribute the target subject is classified into, on the basis of the classification index calculated by the vascular-age calculator 21, and the model selector 60 selects an estimation model corresponding to the applicable attribute as a model optimal for the target subject.

S24 to S30 are performed every time a predicted blood pressure is calculated. Firstly in S24, the image pickup unit 11 captures an image of the target subject. Then in S25, the facial-image acquiring unit 14 acquires a facial image of the target subject from the subject's image captured by the image pickup unit 11.

Then in S26, the facial-image divider 15 sets (divides) a region that is to undergo pulse wave calculation from the facial image extracted by the facial-image acquiring unit 14. Then in S27, the skin region extractor 16 extracts, as skin regions, regions where the skin remains uncovered from the region set by the facial-image divider 15, and the pulse wave calculator 17 calculates a pulse wave for each skin region extracted by the skin region extractor 16.

Then in S28, the pulse-wave-parameter calculator 20 calculates, as a pulse wave parameter, the amount of a pulse waveform characteristic from the pulse wave of each skin region calculated by the pulse-wave acquiring unit 10. Here in S28, the pulse-wave-parameter calculator 20 may calculate a pulse transit time as the pulse wave parameter when there are multiple regions that have undergone pulse wave calculation.

Then in S29, the blood-pressure measuring unit 160 calculates a predicted blood pressure from the pulse wave parameter calculated by the pulse-wave-parameter calculator 20, and from a blood-pressure-estimation model conforming to the subject's attribute selected in S21 through S23. Then in S30, the blood-pressure-measurement outputting unit 170 outputs the resultant predicted blood pressure calculated by the blood-pressure measuring unit 160.

Effects

The second embodiment has described classifying the attribute of the subject H on the basis of an average blood pressure reflecting, for instance, information about the degree to which small peripheral blood vessels have artery hardening (see http://medica.sanyonews.jp/article/4523). The second embodiment has also described blood pressure measurement using a model created for each attribute classification.

This configuration can improve the degree of how much each subject conforms to an estimation model, thus offering a high accurate blood pressure prediction. In this embodiment, the model selector 60 selects a blood-pressure-estimation model optimal for a subject in accordance with his/her sex and with an average blood pressure value calculated from a blood pressure value acquired by the cuff blood-pressure gauge. In some embodiments, further classification may be performed based on, for instance, the actual age and body weight of the subject.

Modification

The second embodiment has described calculating, for each subject H, an average blood pressure reflecting information about artery hardening that occurs in small peripheral blood vessels, by using systolic and diastolic blood pressures acquired in advance by the cuff blood-pressure gauge. The second embodiment has also described classifying the attribute of the subject H on the basis of the average blood pressure, and selecting a suitable model.

The following describes an instance where in addition to an average blood pressure, a pulse pressure that can be calculated from the difference between systolic and diastolic blood pressures is used as a classification index for determining the attribute of a subject.

Although the overall configuration is similar to that in the second embodiment, the vascular-age calculator 21 in this modification has an additional function. The vascular-age calculator 21 calculates an average blood pressure and a pulse pressure from a blood pressure value (SBP and DBP) acquired by the blood-pressure acquiring unit 2. The pulse pressure can be calculated by an expression SBP−DBP. The vascular-age calculator 21 outputs the calculated average blood pressure and pulse pressure to the attribute classifier 23.

An average blood pressure reflects information about artery hardening that occurs in small peripheral blood vessels, and a pulse pressure reflects information about artery hardening that occurs in large blood vessels near the heart (see:http://medica.sanyonews.jp/article/4523). In addition, it is known that artery hardening occurs firstly in small peripheral blood vessels and eventually progresses in large blood vessels along with aging. As such, one's average blood pressure rises firstly (artery hardening in small peripheral blood vessels), and then his/her pulse pressure gradually increases (artery hardening in large blood vessels near the heart) from around after age 50.

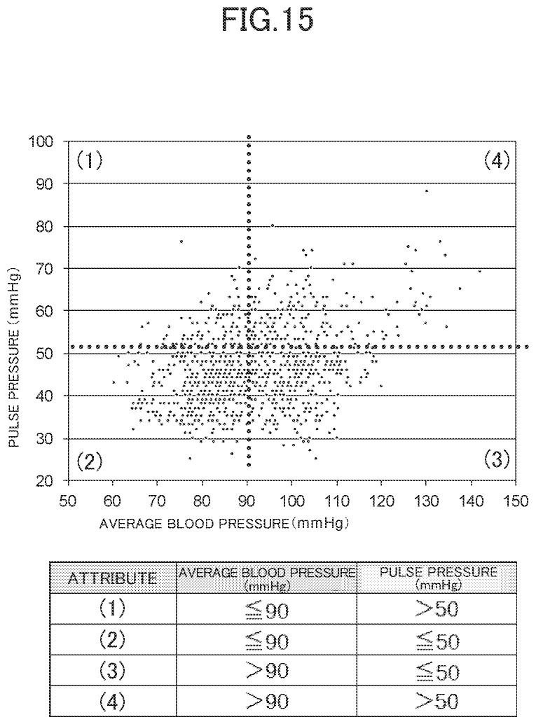

For instance, four attribute classifications are provided in accordance with the volume of average blood pressure and pulse pressure, as illustrated in FIG. 15. The table of FIG. 15 demonstrates that Classification (4), which indicates high average blood pressure and large pulse pressure, has subject's data indicating that artery hardening is in progress further than that of Classification (3), which indicates high average blood pressure and small pulse pressure.

Classification of each target subject based on such average blood pressures and pulse pressures enables blood pressure estimation that reflects the degree of progress of artery hardening and reflects differences in vascular condition (e.g., a location of hardening) between the individuals. Although artery hardening progresses (small blood vessels hardens, followed by large blood vessels) with aging, the degree of its progress differs from one person to another even when they are the same age, depending on their lifestyles until now, such as eating habit and fitness habit. The configuration in this modification enables classification based on information about an actual vascular condition, thereby achieving blood pressure estimation with a model conforming to a target subject, when compared to age-based classification.

In the second embodiment, an average blood pressure and a pulse pressure are calculated based on an SBP and a DBP, acquired by the cuff blood-pressure gauge and are used as an index for attribute classification. In some embodiments, the acquired SBP and DBP per se may be used as a classification index.

Modifications

The second embodiment has described an instance where a user manually inputs, to a smartphone, a blood pressure value for determining the attribute information of each subject.

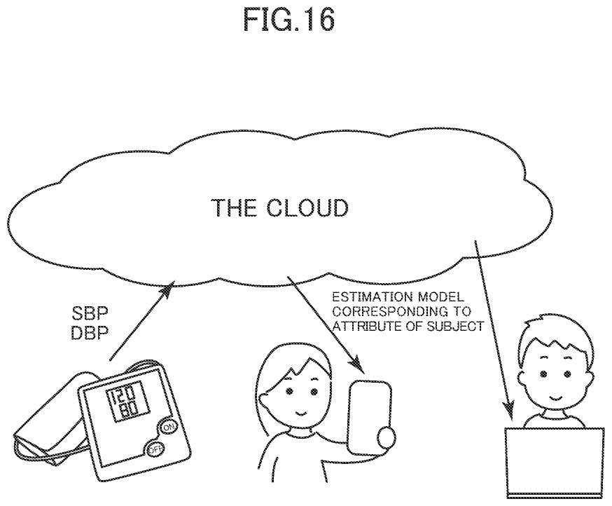

Reference is now made to blood pressure measurement in a hospital, a pharmacy, or other places, and to blood pressure measurement in a medical checkup in a school, a company, or other places. In these cases, the following process steps are performed, as illustrated in FIG. 16: automatically sending an acquired blood pressure value to the cloud (specifically, to a server on the cloud for instance); then, calculating an index necessary for determining the attribute of a subject on the cloud (specifically, in a server on the cloud for instance); then, downloading the subject's attribute and an estimation model corresponding to the attribute to subject's hardware (e.g., a smartphone or a PC), thus performing model selection and model update.

When the blood-pressure acquiring unit 2 is hardware that can communicate with the cloud, a blood pressure value acquired by the blood-pressure acquiring unit 2 is associated with information from which an individual subject can be identified, such as his/her name, ID, and facial image (face recognition), and the associated blood pressure value is sent to the cloud.

When a measurement result in a medical checkup in a school or a company is used in selecting and updating a model for a subject, a model optimal for the subject is selected on the basis of measurement values obtained in the medical checkup, in cooperation with a database where these measurement values are aggregated.

When communicably connected to the cloud (specifically, to a server on the cloud for instance), the model selector 60 disposed inside the blood-pressure measuring device 1 may periodically automatically update a model corresponding to the attribute of a subject, or this model selector 60 may download a model corresponding to an attribute optimal for the subject from the cloud (specifically, from a server on the cloud) in response to a subject's request for model update.

Many companies and schools oblige employees and students to take a medical checkup at least once a year. Using a measurement result in the checkup can thus update an optimal estimation model for a target subject at least once a year. This enables blood pressure prediction using an estimation model that periodically reflects the vascular condition of the target subject, thereby keeping accuracy at a certain level without degradation.

Using a measurement result in a medical checkup, a hospital or other places allows those who have no cuff blood-pressure gauge in their homes to accurately measure daily fluctuations in their blood pressure using a smartphone, a PC and other equipment. In a blood test on a medical checkup, an index indicating, for instance, blood viscosity may be used as an index for attribute classification.

After a lapse of a predetermined period for instance, from the calculation of the latest classification index (average blood pressure and/or pulse pressure) for a subject, the blood-pressure measuring device 1 may send a notification to the subject to prompt model update. This enables selection of a model based on the subject's current vascular condition, and blood pressure measurement using the selected model, thereby achieving a certain accuracy level.

### Third Embodiment

(1) A subject H is not limited to a human. The subject H needs to be a target to which the blood-pressure measurement method according to one aspect of the present disclosure is applicable. For instance, the subject H may be an animal, such as a dog or a cat.

(2) An ROI is not limited to a face. The ROI needs to be the body surface of a living body through which its pulse wave can be obtained. Other examples of the ROI include a neck, a chest, and a palm. However, the ROI is preferably a face. Using an IMG (facial image) can reduce a burden on the subject H during blood pressure measurement. That is, using an IMG facilitates measuring the blood pressure of the subject H who is under natural conditions (or is relaxed).

(3) The blood-pressure measuring unit 160 does not necessarily have to calculate a P using only one model for blood pressure measurement. That is, the model selector 60 may select a plurality of models for blood pressure measurement. By way of example, the blood-pressure measuring unit 160 calculates a plurality of blood pressures by using a plurality of respective models for blood pressure measurement. The blood-pressure measuring unit 160 in this case may calculate a representative value (e.g., an average or median value) of these blood pressures and output the representative value as the P.

(4) Attribute information does not necessarily have to be acquired through image analysis. For instance, a contact sensor can be used as the attribute-information acquiring unit. The sensor needs to be designed not to make the subject H feel restrained as much as possible. As described above, the blood-pressure measuring device 1 may be a contact blood-pressure measuring device.

(5) An estimation model is not limited to a linear model. The model creating unit 30 may create a non-linear model (a calculation model expressed by a non-linear function) through regression analysis. The model creating unit 30 may also create an estimation model by using a method other than the method of least squares. For instance, the model creating unit 30 may create a linear model through Lasso regression introducing L1 regularization. Using Lasso regression can create a linear model that reflects over-learning prevention.

(6) As described above, an evaluation index (PI) of an estimation model needs to be a parameter that is calculated based on a BPe and a BPm. The PI is hence not necessarily limited to an error-related parameter. Examples of the PI usable include (i) an adjusted index of determination and (ii) the Akaike's information criteria (AIC).

(7) An index for evaluating the signal quality of a pulse wave is not limited to an SNR. For instance, the pixel value of each skin region can be used as this evaluation index.

(8) The model storage 55 needs to be communicably connected to the blood-pressure measuring device 1. For instance, the model storage 55 may be a server external to the blood-pressure measuring device 1. In this way, the model storage 55 does not necessarily have to be disposed within the blood-pressure measuring device 1. The model storage 55 does not also necessarily have to be disposed within the model setting device 100.

The model storage 55 can be omitted. The model selector 60 in this case needs to acquire each estimation model directly from the model creating unit 30. The model selector 60 also needs to acquire each PI directly from the model evaluating unit 40. However, the model storage 55 is preferably provided in order for the blood-pressure measuring device 1 to speedily measure a P.

(9) The model setting device 100 needs to be communicably connected to the blood-pressure measuring device 1. For instance, the model setting device 100 may be a server external to the blood-pressure measuring device 1. In this way, the model setting device 100 does not necessarily have to be disposed within the blood-pressure measuring device 1. The blood-pressure measuring device 1 may be thus implemented by a publicly known information processer (e.g., a smartphone, a tablet, or a personal computer).

### Fourth Embodiment

The control blocks of the blood-pressure measuring device 1 (in particular, the model setting device 100, the model selector 60, the pulse-wave-signal quality evaluating unit 150, and the blood-pressure measuring unit 160) may be implemented by a logic circuit (hardware) formed in, for instance, an integrated circuit (IC chip) or by software.

For software, the blood-pressure measuring device 1 includes a computer that executes commands of a program, which is software that implements each function. The computer includes, for instance, at least one processor (controller) and at least one computer-readable recording medium storing the program. The processor in the computer reads the program from the recording medium and executes the program, thus achieving the object of one aspect of the present disclosure. An example of the processor usable is a central processing unit (CPU). An example of the recording medium usable is a non-transitory tangible medium, including a read-only memory (ROM), a tape, a disc, a card, a semiconductor memory, and a programmable logic circuit. The computer may also include, for instance, a random access memory (RAM) that develops the program. The program may be supplied to the computer via any transmission medium (e.g., a communication network or a broadcast wave) capable of transmitting the program. One aspect of the present disclosure can be implemented also in the form of a data signal in which the program is embodied by electronic transmission and that is embedded in a carrier wave.

### ADDITIONAL REMARKS

The present disclosure is not limited to the foregoing embodiments. Various modifications can be devised within the scope of the claims. In addition, an embodiment that is obtained in combination, as appropriate, with the technical means disclosed in the respective embodiments is also included in the technical scope of one aspect of the present disclosure. Furthermore, combining the technical means disclosed in the respective embodiments can offer a new technical feature.

1. A blood-pressure measuring device that measures a first blood pressure of a living body on the basis of a pulse wave of the living body, the blood-pressure measuring device comprising:
a pulse-wave acquiring unit configured to acquire at least one pulse wave in a predetermined region on a body surface of the living body;
a pulse-wave-parameter calculator configured to calculate at least one pulse wave parameter based on the at least one pulse wave;
an attribute-information acquiring unit configured to acquire attribute information that relates to a vascular condition of the living body; and
an attribute classifier configured to classify an attribute of the living body in accordance with the attribute information,
wherein the blood-pressure measuring device is communicably connected to a model storage storing in advance one or more blood-pressure-estimation models that are used for estimating the first blood pressure based on a result of classification of the attribute, and
the blood-pressure measuring device further comprises
a first-blood-pressure measuring unit configured to calculate the first blood pressure based on the at least one pulse wave parameter by using the one or more blood-pressure-estimation models based on the result of classification of the attribute.

2. The blood-pressure measuring device according to claim 1, wherein
the model storage further stores in advance results of evaluation of the one or more respective blood-pressure-estimation models, and
the first-blood-pressure measuring unit calculates the first blood pressure based on the at least one pulse wave parameter by using a measurement model, the measurement model being selected from among the one or more blood-pressure-estimation models by a model selector in accordance with the results of evaluation of the one or more respective blood-pressure-estimation models and being based on the result of classification of the attribute, the model selector being configured to select the measurement model for calculating the first blood pressure.

3. The blood-pressure measuring device according to claim 1, wherein
the attribute classifier classifies the attribute into N different patterns comprising first to Nth attributes, where N is an integer equal to or greater than two, and
the model storage stores in advance at least one kth model, where k is an integer ranging from one to N inclusive, the at least one kth model being included in the one or more blood-pressure-estimation models corresponding to a kth attribute.

4. The blood-pressure measuring device according to claim 1, wherein
the attribute-information acquiring unit acquires the attribute information by analyzing an image including an image of the predetermined region.

5. The blood-pressure measuring device according to claim 1, wherein
the attribute-information acquiring unit acquires the attribute information by analyzing the at least one pulse wave.

6. The blood-pressure measuring device according to claim 1, wherein
the attribute-information acquiring unit acquires the attribute information by referring to a blood pressure value obtained from a contact blood-pressure gauge.

7. The blood-pressure measuring device according to claim 6, wherein
the attribute information includes information indicating an average blood pressure of the living body.

8. The blood-pressure measuring device according to claim 6, wherein the attribute information includes information indicating a pulse pressure of the living body.

9. The blood-pressure measuring device according to claim 6, wherein
the attribute-information acquiring unit acquires the attribute information via a cloud.

10. The blood-pressure measuring device according to claim 6, wherein
the attribute information is calculated on a cloud.

11. The blood-pressure measuring device according to claim 1, wherein
the attribute information includes information indicating a vascular age of the living body or an apparent age of the living body.

12. The blood-pressure measuring device according to claim 1, wherein
the attribute information includes information indicating an amount of a waveform characteristic of the at least one pulse wave.

13. The blood-pressure measuring device according to claim 1, wherein
the attribute information includes information indicating whether a swelling is found in the predetermined region.

14. The blood-pressure measuring device according to claim 1, wherein
the attribute information further includes information indicating sex of the living body.

15. The blood-pressure measuring device according to claim 1, wherein
the predetermined region is a face of the living body.

16. A model setting device communicably connected to a blood-pressure measuring device that measures a first blood pressure of a living body on the basis of a pulse wave of the living body, the model setting device comprising:
a second-blood-pressure measuring unit configured to measure a second blood pressure of the living body;
a pulse-wave acquiring unit configured to acquire at least one pulse wave in a predetermined region on a body surface of the living body;
a pulse-wave-parameter calculator configured to calculate at least one pulse wave parameter based on the at least one pulse wave;
an attribute-information acquiring unit configured to acquire attribute information that relates to a vascular condition of the living body; and
an attribute classifier configured to classify an attribute of the living body in accordance with the attribute information,
wherein the model setting device is communicably connected to a model storage capable of storing one or more blood-pressure-estimation models that are used for estimating the first blood pressure based on a result of classification of the attribute, and
the model setting device further comprises
a model creating unit configured to create the one or more blood-pressure-estimation models based on the at least one pulse wave parameter and the second blood pressure, and store the one or more blood-pressure-estimation models in the model storage.

17. The model setting device according to claim 16, further comprising
a model evaluating unit configured to evaluate the one or more blood-pressure-estimation models individually and store results of evaluation of the one or more respective blood-pressure-estimation models in the model storage.

18. The model setting device according to claim 16, wherein
the attribute classifier classifies the attribute into N different patterns comprising first to Nth attributes, where N is an integer equal to or greater than two, and
the model creating unit includes
a kth-model creating unit configured to create at least one kth model and store the at least one kth model in the model storage, where k is an integer ranging from one to N inclusive, the at least one kth model being included in the one or more blood-pressure-estimation models corresponding to a kth attribute.

19. A blood-pressure measurement method using a blood-pressure measuring device that measures a first blood pressure of a living body on the basis of a pulse wave of the living body, the method comprising the steps of:
acquiring at least one pulse wave in a predetermined region on a body surface of the living body;
calculating at least one pulse wave parameter based on the at least one pulse wave;
acquiring attribute information that relates to a vascular condition of the living body; and
classifying an attribute of the living body in accordance with the attribute information,
wherein the blood-pressure measuring device is communicably connected to a model storage storing in advance one or more blood-pressure-estimation models that are used for estimating the first blood pressure based on a result of classification of the attribute, and
the method further comprises
a step of calculating the first blood pressure based on the at least one pulse wave parameter by using the one or more blood-pressure-estimation models based on the result of classification of the attribute.

20. The blood-pressure measuring device according to claim 6, wherein
the attribute-information acquiring unit acquires the attribute information at least once before measurement of the first blood pressure in accordance with a blood pressure value obtained from the contact blood-pressure gauge.

21. The blood-pressure measuring device according to claim 1, wherein
the attribute information includes information about a degree of artery hardening of a blood vessel of the living body.
